(19) United States
(12) Patent Application Publication
(10) Pub. No.: US 2009/0138061 A1
(43) Pub. Date: May 28, 2009

(54) Stimulator For The Control of a Bodily Function

(75) Inventors: Anthony C.N. Stephens, New South Wales (AU); Linda Laidlaw, New South Wales (AU); Narelle Bramich, Victoria (AU)

Correspondence Address: JONES DAY, 222 EAST 41ST ST, NEW YORK, NY, 10017, US

(73) Assignee: Continence Control Systems International Pty Limit, Chatswood, NSW (AU)

(21) Appl. No.: 12/087,792

(22) PCT Filed: Jan. 16, 2007
(86) PCT No.: PCT/AU2007/000027

§ 371 Date: Oct. 7, 2008

(30) Foreign Application Priority Data
Jan. 16, 2006 (AU) 2006900210

(51) Int. Cl. A61N 1/36; A61N 1/08

(52) U.S. Cl. 607/41

(57) ABSTRACT

The present invention provides a device for the stimulation of smooth muscle tissue. The device includes a stimulator arranged to provide a signal to the smooth muscle tissue to control response of the smooth muscle tissue, and an interface arranged to allow programming of a controller of the stimulator. The interface may interact with an external controller. The device finds use in controlling smooth muscle tissue, such as a neosphincter, for the control of urinary or faecal incontinence in a patient.

### CROSS REFERENCE TO RELATED APPLICATIONS

The Applicant of the present application has previously filed a number of patent applications for aspects of an implantable device arranged to control bodily functions. These applications include:
PCT Application No. PCT/AU2005/001698, entitled “AN IMPLANTABLE ELECTRODE ARRANGEMENT”, filed on 8 Nov. 2005;
Australian Provisional Application No. 2005900957, entitled “IMPROVED METHOD AND DEVICE FOR MANAGING URINARY INCONTINENCE”, filed on 2 Mar. 2005;
Australian Provisional Application No. 2005904830, entitled “AN IMPLANT FOR MANAGING A MEDICAL CONDITION”, filed on 2 Sep. 2005;
Australian Provisional Application No. 2005904382, entitled “METHOD AND APPARATUS FOR CONTROLLING BLADDER FUNCTION”, filed on 15 Aug. 2005;
Australian Provisional Application No. 2005905673, entitled “A METHOD AND APPARATUS FOR TREATING ANAL INCONTINENCE”, filed on 14 Oct. 2005; and
Australian Provisional Application No. 2005905672, entitled “A METHOD AND APPARATUS FOR TREATING A HEART CONDITION”, filed on 14 Oct. 2005.

All the abovementioned applications are herein incorporated by reference.

### FIELD OF THE INVENTION

The present invention generally relates to a stimulator for the control of a bodily function, and specifically, but not exclusively, to a controller and stimulator for smooth muscle tissue, such as a neosphincter, which may be formed as a ring of smooth muscle tissue or any other mechanical configuration to address a deficiency in a bodily function.

### BACKGROUND OF THE INVENTION

Prior art relating to implantable medical devices has been associated with the development of cardiac pacemakers, first disclosed by Wilson Greatbatch in U.S. Pat. No. 3,057,056 entitled “Medical Cardiac Pacemaker” which issued in 1962. Since that time there has been an evolution of the technology—directed to the cardiac application but providing flexibility to this underlying technology. One such example is the introduction of programmable parameters (U.S. Pat. No. 3,805,796 in the name of Reese Terry, Jr et al. “Implantable Cardiac Pacemaker having Adjustable Operating Parameters, issued in 1974).

With the introduction of low power electronics (for example, CMOS) and microprocessors a flexible stimulator that can be modified externally has been developed, using a separate instrument to interact and re-program the operating parameters of the implanted system. For example, see U.S. Pat. No. 4,440,173 to LC Hudziak et al., “Programmable Body Stimulation System” issued 1984; U.S. Pat. No. 4,424,812 to AF Lesnick “Implantable Externally Programmable Microprocessor-Controller Tissue Stimulator” issued 1984; U.S. Pat. No. 4,432,360 to VE Mumford “Interactive Programmer for Biomedical Implantable Devices” issued 1984; U.S. Pat. No. 4,515,159 to RS McDonald et al. and “Digital Cardiac Pacemaker with Rate Limit Means”, issued 1985.

In some cases, the underlying stimulation system has been developed to allow the device to be used for multiple purposes. For example, U.S. Pat. No. 4,592,360 to AF Lesnick “Implantable Externally Programmable Microprocessor-controlled Tissue Stimulator” issued 1986.

One such application is to provide stimulation of the nerves of the bladder. U.S. Pat. No. 4,607,639 to EA Tanagho “Method and System for Controlling Bladder Evacuation” issued 1986 describes a method stimulating the sacral nerves to cause contraction of the bladder, and by cutting the sensory fibres in the sacral nerves, preventing simultaneous activation of the sphincter, thus enabling the bladder to empty. U.S. Pat. No. 4,569,351 to PC Tang “Apparatus and Method for Stimulating Micturition and Certain Muscles In Paraplegic Mammals” issued 1986 describes an improvement in providing intermittent electrical stimulation delivered to the spinal canal in the vicinity of the sacral roots to cause contraction of the detrusor and raise bladder pressure. Following cessation of stimulation, the raised bladder pressure can result in the bladder emptying.

Additional methods have been proposed including selective stimulation of the mixed nerves (for example W Grill et al., U.S. Pat. No. 6,907,293 B2 issued 2005 and patent applications, 20050060005 A1 and 20050222636 A1), stimulating a skeletal muscle placed over the bladder to cause bladder emptying on activation of the transplanted skeletal muscle (U.S. Pat. No. 5,752,978 to M Chancellor, “Detrusor Myoplasty and Neuromuscular Electrical Stimulation”, issued 1998) and electrical stimulation of the pelvic floor and/or bladder sphincter anatomy in response to pressure changes (for example U.S. Pat. Nos. 6,896,651, 6,862,480, 6,652,449, 6,354,991 to Gross et al., issued 2005 and US patent applications US2005113881A1 and US2005049648A1).

### SUMMARY OF THE INVENTION

In contrast to the prior art, aspects of the present invention define an implantable stimulation system that stimulates smooth muscle tissue that is transplanted to address a deficiency in a bodily function.

In one aspect, the present invention provides a device for the stimulation of smooth muscle tissue, comprising a stimulator arranged to provide a signal to the smooth muscle tissue to control response of the smooth muscle tissue, and an interface arranged to allow programming of a controller of the stimulator.

The signal may be passed from the implanted stimulator to the smooth muscle tissue via a stimulation lead, such as an electrode. The device may be implantable within a patient and the smooth muscle tissue may be a sphincter or a neosphincter.

The signal delivered to the smooth muscle tissue may be a symmetric or an asymmetric waveform, which may be biphasic. Where the waveform is biphasic, the stimulator may be arranged to introduce a time delay between each of the two phases of the waveform. The delay may be in the range from approximately 0 to 100 milliseconds.

The smooth muscle tissue may be positioned to cause a mechanical change in configuration on activation by the electrical stimulation, to address a deficiency in a bodily function.

The signal applied to the smooth muscle tissue may be greater than 0 mA but less than or equal to 25 mA. Generally, the stimulator lead will have an impedance of less than 2 kilohms.

The device may further comprise sensors arranged to monitor the response of the smooth muscle tissue and/or sensors arranged to monitor a bodily function of the patient.

The bodily function may be the fullness of the bladder of the patient or the perception of fullness by the patient, the fullness of the rectum of the patient, the commencement and/or cessation of urination by the patient and/or the commencement and/or cessation of defecation by the patient. Sensors relevant to other bodily functions could also be applied in combination with the device. The addition of one or more relevant sensors may facilitate the use of the system with cognitively impaired, aged and or demented patients in which the patient is unable to initiate action themselves in response to feedback. Alternatively, a carer or supervisory clinician may initiate specific bodily functions (for example urination or defecation) at an appropriate and convenient time when care is available, thereby facilitating management of the patient.

The device may further comprise storage means arranged to store data collected from the sensors, including events associated with a patient initiated action, or automatically by the sensor (for example, if the sensor detects a change in pressure, volume or other parameter above a pre-defined threshold level).

The power for the device may be an in-built battery, a rechargeable battery, or a charge storing device such as a capacitor or some other means for storage and provision of energy.

The power switch for the device may be magnetically controlled, or it may be a device arranged to respond to an electromagnetic signal.

The device may further comprise means to provide feedback to a patient on the status of the device via an audible signal or a tactile signal from the device itself, or a visual, audible or tactile signal from an external controller.

The feedback may alert the patient to any one or any combination of the power status of the device, the power delivered by the device, or the fullness of the bladder or rectum, or the sense of fullness of one or both organs, or related anatomical changes (for example, abdominal pressure or muscle activation sensed by electromyogram) related to this fullness which can modify stimulation to the smooth muscle tissue.

In another aspect, the present invention provides a device for the stimulation of smooth muscle tissue, comprising a stimulator arranged to provide a signal to the smooth muscle tissue to control the response of the smooth muscle tissue, and means to provide feedback to a patient on the status of a controller of the stimulator.

In another aspect, the present invention provides a device for use with a stimulator arranged to provide a signal to smooth muscle tissue to control the response of the smooth muscle tissue, the device comprising at least one sensor arranged to monitor a bodily function of a patient.

The at least one sensor may be arranged to provide feedback to a patient of a bodily function associated with a sphincter, a neosphincter or smooth muscle tissue that is transplanted in a particular configuration to address a deficiency in bodily function.

In another aspect, the present invention provides a system for the stimulation of smooth muscle tissue, comprising a device in accordance with the other aspects of the invention and an external controller arranged to communicate with the interface.

The external controller may be arranged to upload control instructions to the stimulator controller or to a storage means. The external controller may also be arranged to download data from a storage means.

In another aspect, the present invention provides a device for the stimulation of smooth muscle tissue, comprising a stimulator arranged to provide a signal arranged to stimulate the smooth muscle tissue, and a controller arranged to control the signal provided by the stimulator in a manner which influences the innervation of the smooth muscle tissue.

In another aspect, the present invention provides a method for calibrating a device for the stimulation of smooth muscle tissue, comprising the steps of measuring the impedance of a stimulation lead arranged to provide a stimulus signal to the smooth muscle tissue, measuring the response of the smooth muscle tissue, and, if necessary, adjusting the signal.

In another aspect, the present invention provides a method of stimulating a smooth muscle tissue, comprising the steps of utilising a device in accordance with another aspect of the invention to control the smooth muscle tissue, by applying a signal to the smooth muscle tissue to cause the smooth muscle tissue to contract.

In another aspect, the present invention provides a method for stimulating the pelvic floor, comprising the steps of utilising a device in accordance with another aspect of the invention to stimulate the pelvic floor to cause the pelvic floor muscle to contract, thereby strengthening the pelvic floor.

In another aspect, the present invention provides a device in accordance with another aspect of the invention, comprising a plurality of stimulators to stimulate a plurality of discrete smooth muscle neosphincters.

The plurality of bodily functions includes at least one of the control of urine flow from the bladder and the control of stools and/or flatus from the rectum.

### DESCRIPTION OF THE DRAWINGS

Features and advantages of the present invention will become apparent from the following description of embodiments therefore, by way of example only, with reference to the accompanying drawings, in which:

FIGS. 1, 2 and 3 are block diagrams which depict alternative embodiments of control systems for a sphincter, in accordance with an embodiment of the present invention;

FIGS. 4 and 5 are flow charts which depict a methodology for treating incontinence, utilising an embodiment of the present invention to treat different medical conditions;

FIGS. 6 and 7 are flowcharts depicting a methodology for optimizing the stimulation parameters of a device in accordance with an embodiment of the present invention; and

FIGS. 8, 9 and 10 are flowcharts which depict control algorithms for a controller used to control a sphincter, in accordance with an embodiment of the present invention.

### DESCRIPTION OF SPECIFIC EMBODIMENTS

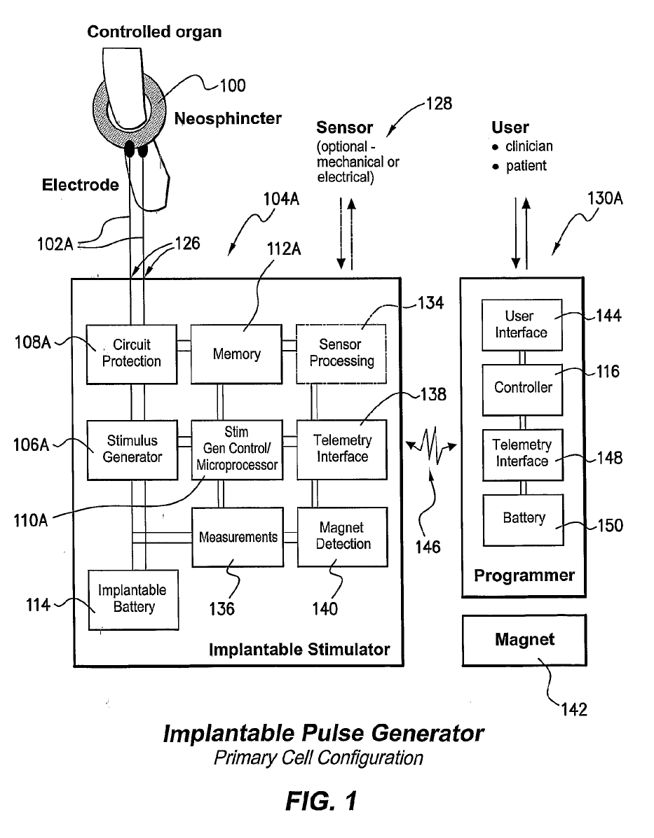

Referring to FIGS. 1, 2 and 3, there are shown three different embodiments of a sphincter control system including an implantable stimulator system which interfaces with a sphincter via an electrode. The implantable stimulator system is arranged to communicate with an external controller.

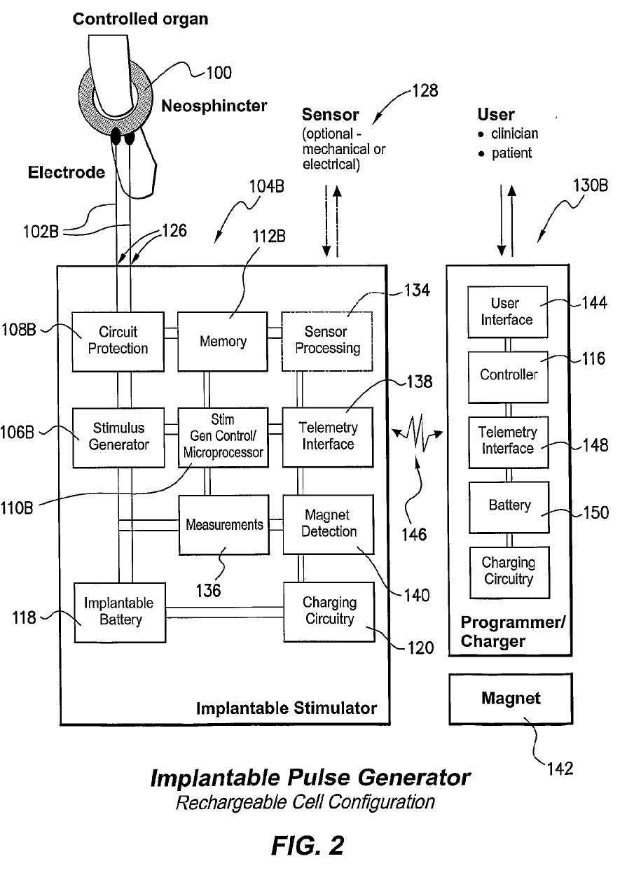

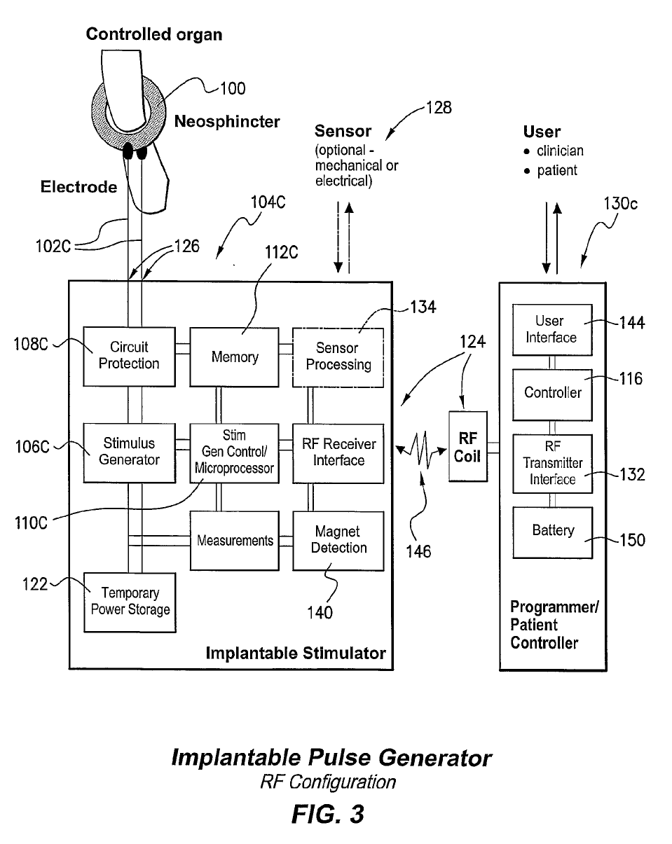

Each sphincter control system described herein is connected to a neosphincter 100 via a means of delivering electrical stimulation to the neosphincter, such as stimulation leads (which, in one embodiment, is an electrode) 102A, 102B or 102C and may include one or more additional means of obtaining feedback for the system, such as a sensor. Each of the sphincter control systems 104A (FIG. 1), 104B (FIG. 2) and 104C (FIG. 3) comprise internal components required to provide the functions necessitated by a sphincter control system, including a stimulus generator or other means of creating stimulation pulses 106A, 106B and 106C in connection with the electrode 102 through circuit protection or other means 108A, 108B, 108C to prevent damage to the stimulation control system's internal circuitry caused by externally applied therapy, such as a defibrillation pulse. In each embodiment, the stimulus generator 106A, 106B or 106C is controlled by a stimulus generator controller 110A, 110B or 110C (respectively) which each contains memory or other means of storing data 112A, 112B or 112C (respectively), which may be utilised to hold control algorithms. The stimulus generator controller provides a means by which specific operating parameters are varied either by direct user or clinician command, or by algorithms which dynamically change these parameters from either values provided by the user or clinician, or using information derived from other input, such as one or more sensors.

The sphincter control systems may utilise any one of a number of power technologies to operate the control system and to generate the necessary waveforms to control the sphincter such as transmission of Radio Frequency electromagnetic radiation that is stored by the implant, or by using an implantable battery, that may be housed within the implantable part of the sphincter control system.

Referring now to FIG. 1, there is described an embodiment where the stimulator is powered by a primary implantable battery 114 which provides power to the stimulator. The battery will generally have a useful lifetime in the order of 4-6 years after first implant depending on the extent of use and required stimulation characteristics (for example, pulse amplitude or frequency) to stimulate the smooth muscle tissue to address a deficiency in bodily function. When the battery level falls to an unacceptably low level, the stimulator is replaced with a new stimulator that has a fresh (undepleted) cell. An example of a suitable cell chemistry for an implantable primary cell is Lithium cell although other battery chemistries are possible. In some embodiments, a separate power source usually a commercial battery, such as one or more AA or AAA battery, a custom battery (often also rechargeable) or a medically isolated power supply that is connected to the mains power, provides power to the programmer or control circuitry. In sphincter control systems incorporating an implantable battery as a power source, the programmer only requires power when it is communicating with the implantable stimulator. This may occur where the clinician varies the stimulus intensity, or where a patient or a clinician turns the system on or off, changes any parameters or interrogates the implantable stimulator to obtain data logs or perform measurements. The battery may also operate the telemetry communication module when data is downloaded from the stimulator for analysis.

FIG. 2 depicts an embodiment, where a Rechargeable Cell Configuration is utilized. The embodiment of FIG. 2 includes a Rechargeable Cell Configuration, wherein the implanted battery 118 may be periodically recharged by using changing circuitry 120 (such as a radio frequency link) to “top-up” the battery. The interval between recharging depends on the amount of use. Over time, the battery may lose the ability to retain a suitable charge, requiring a patient or a clinician to recharge the battery more frequently, or eventually replace the battery. This may involve replacement of the entire stimulator, in a similar manner to the Primary Cell System of FIG. 1. In addition to being rechargeable, the rechargeable battery may be considerably smaller (for example, half the volume of a primary battery) and therefore reduce the size of the stimulator system. Additionally, some types of rechargeable batteries can source currents capable of providing intense stimulation (for example, in some cases, a current pulse of greater than 10 mA may be generated).

Referring to FIG. 3, there is shown a further embodiment of the present invention. The third embodiment utilizes an RF Configuration as a power supply. In this system architecture, there is no battery in the stimulator. Rather, there is included a means for temporary power storage 122 (for example, a capacitor). Electromagnetic energy is continuously transferred by a Radio Frequency link 124 from an external programmer/controller while the system is on. The power for the entire system is thus provided externally, and the controller may utilize commercial batteries (for example AAA or AA size), or rechargeable non-implantable batteries (such as Lithium Ion batteries). The advantage of the RF configuration is that higher intensity stimuli can be delivered (in some cases greater than 10 mA) and additionally, the system can be powered from mains power using a medically isolated power supply.

In more detail, the Stimulator provides one or more channels 126 of electrical stimulation to adjust activation of the neosphincter. In an alternate embodiment, the stimulator may also provide neuromodulation to inhibit urge events. In a further embodiment, the Stimulator may provide stimulus control to more than one sphincter that has been implanted to assist in a bodily function in the one subject. As one example, a user with severe urinary incontinence may require the use of two sphincters, each controlled by the sphincter control system to achieve urinary continence. Alternatively, a user with both urinary and faecal incontinence could use one implanted stimulator system to enable control of both bodily functions.

Optionally, the system may include one or more sensors 128 to provide input to enable automatic control of relevant functions. The sensor 128 will interface with the Sensor Processing module in the stimulator. The stimulator may also collect sensor data or acquires electrical data on the activity of neosphincter.

The system also interfaces with an external instrument (controller) 130A (FIG. 1), 130B (FIG. 3) and 130C (FIG. 3) including controller circuitry 116 which enables the clinician or patient to control the implantable stimulator by programming parameter values (for example the stimulus pulse amplitude, the pulse width, and/or the frequency). The Clinician/Patient Controller can also be used to initiate measurements (for example, stimulation lead integrity and battery status). Data that has been acquired by the implantable stimulator can also be downloaded to the Clinician/Patient Controller. For example when the system is first activated, the clinician uses the external Controller to set the stimulation parameters to 0.1 ms, 1 Hz and 2 mA. The continence state of the patient is then assessed using either cystoscopy, urodynamics or some other diagnostic test (e.g. pad weight test). If leakage is still apparent the clinician uses the Controller to increase the level of stimulation until continence is achieved (eg this may occur with stimulus parameters set to 0.4 ms, 2 Hz, 4 mA). When the stimulus parameters have been set the clinician may then download stored information (eg patient identifier, current and previously tested stimulus parameter values, lead impedance) from the implantable stimulator using the Controller and additional, store them onto their PC for future referral.

For implantable systems incorporating a rechargeable implantable battery, the Programmer/Patient Controller may include the necessary charge storage and RF transmission circuitry 132 to enable the implanted cell to be recharged.

It will be understood that two different external instruments may be made available. A first instrument with basic functionality may be made available to the patient. For example, the patient may have a controller which only allows the patient to activate or deactivate the neosphincter. The controller may also include a basic display or warning system, to notify the patient of a particular set of conditions, such as low battery, bladder fullness, etc. In one embodiment, the controller may be a small handheld instrument (for example, similar to a motor vehicle key which incorporates one or two push buttons and a radiofrequency transmitter and receiver which can remotely enable and disable a car's security alarm as the driver approaches). Optionally, a larger instrument could also incorporate a small screen to display status of the sphincter control system. In another embodiment, the controller is a magnet. The magnet is utilized for implantable systems which incorporate an implantable battery and can operate autonomously. The magnet is utilized by the patient to control the implanted system (for example, to switch the system on or off), or as a means for the clinician controlling the system in an emergency where no other controller is available. In the embodiment utilizing an RF coil as a power source, the RF coil is required to transfer electromagnetic radiation to the implantable stimulator to provide power and also as a data transmitter. In such systems the user can temporarily stop stimulation by removing the RF coil overlying the stimulator.

A second instrument with additional functionality may be made available to the clinician. The instrument may be capable of collecting data from the controller, to allow the clinician to identify problems (such as stimulation lead integrity, stimulus output errors, logged data on system function and user-initiated operations, and other diagnostic tests to confirm the overall function of the system). The controller may also allow the clinician to change the programming of the stimulator, in the event that any problems are identified.

Referring to the stimulator module in more detail, the functional modules which comprise a stimulator module include a means of generating charge-balanced biphasic stimulus waveforms (that is, electrical pulses in which there is ideally no net direct current delivered to the patient). The waveforms may be symmetric (that is, each phase of the biphasic pulse having similar duration) or asymmetric (in which one phase may have a much longer pulse width (for example up to 10 or 20 times), as the first phase). Different waveforms are utilized in different applications and the stimulator may be programmable to generate either type of pulses or a combination of asymmetric and symmetric pulses. This allows the same type of stimulator to be utilized for different applications within the human body. For example, symmetric pulses are utilized in cochlear implants, whereas asymmetric waveforms find a particular application in cardiac applications. In the case of cochlear implants, constant current symmetric biphasic pulses are delivered as this waveform is thought to more effective than other waveforms to stimulate the auditory nerve. In cardiac applications simpler stimulation circuits may be employed which provide an asymmetric biphasic waveform pulse and which require fewer components for implementation, decreasing the size of the pacemaker. A stimulator may be configured to provide more appropriate waveforms on separate channels, depending on the target application. Additionally, the stimulation circuitry may also apply a delay between the two phases of a symmetric or asymmetric biphasic waveform pulses (for example from approximately 10 microseconds to 100 milliseconds).

The stimulator module also includes, in one embodiment, a storage capacitor which provides a source of charge, and additional capacitors that are temporarily switched across the patient load to provide an exponential waveform consistent with a capacitor discharge through a predominantly resistive load. While biphasic waveform pulses have been used routinely in many applications, other waveform shapes may be utilised in the stimulation of tissue for various purposes.

The module also incorporates a stimulator (microprocessor), also referred to as the “Stin Gen Control” in FIGS. 1, 2 and 3. The microprocessor can utilize pre-programmed values (i.e. firmware), or can also “learn” by receiving input from sensors. In the simplest embodiment, the microprocessor controls the time at which a stimulus is delivered, and the specific waveform which is used to excite the tissue. The inputs may be received in electrical or mechanical form, depending on the particular stimulator application. In the embodiment that includes capacitors as an energy storage device, the microprocessor supplies the control signals to the switches that control when and how long the storage capacitors are connected to the load. In other words, the microprocessor is utilized to not only control the signals to the electrode and the sphincter, but also to control other functions, such as controlling energy usage to prolong battery life. Where the microprocessor is utilized to control multiple functions, information can be stored in memory if required.

The microprocessor may also receive information from the Sensor Processing module 134 (if a sensor input is being used to modify stimulation), and/or from the Measurements Module 136 to store data received into the memory module so that a clinician may access the data at a later date for download and off-line analysis.

The Measurements module 136 is used to perform various diagnostic and integrity measurements of either the patient, a sensor if present, and other functional aspects of the sphincter control system. In one embodiment, the measurements block includes specific analog circuitry to enable Analog to Digital Conversion (ADC) for input of the data for processing by a digital microcontroller or microprocessor included in the implantable stimulator, although other signal processing techniques are possible which convert an analog level (e.g. a voltage level on a resistor or capacitor) to a digital signal that can be utilized by a controller or microprocessor. For example, the ADC input for data processing can identify the sensor status when the user perceives bladder fullness, or other changes in related anatomical structures that would signify similar physiological status.

The measurements module also includes circuitry to enable measurement of voltages to confirm system function. One example is to measure a storage capacitor prior to delivery of a pulse, and immediately after delivery of a pulse, or at any other time, to provide an estimate of stimulation lead impedance, which is then saved in memory 112 to confirm that stimulation is being delivered to the stimulator's output. Typical ranges of lead impedance range from 300 ohms to 2000 ohms. This data can be used by the clinician as diagnostic information which can be analysed to determine the integrity of the stimulation lead.

In embodiments which utilize an implantable battery, a voltage measurement of the battery voltage under open circuit and under load can also be taken to provide an indication of useful operating life as the internal resistance of many batteries rise as the cell depletes, with the internal resistance dependent on the specific battery's chemistry. This may be saved as data for later analysis by a clinician, and also may be analysed by the microprocessor so that a warning signal may be sent to the patient if it is determined that the battery is low and needs replacement.

The Measurements module may also incorporate further sensors 128 to enable other diagnostic tests to assess the effectiveness of stimulation.

In one example, the sensor is a piezoelectric element utilized to sense bladder fullness, in which deformation of the element results in a change in electrical impedance of the element. The Measurements module performs the electrical measurements necessary to measure the change in shape of the sensor as an indicator of bladder fullness. For each patient, such a sensor may require calibration to identify the sensor input associated with the fullness of the bladder, or the user's perception of that fullness or other sensor parameter. In one embodiment, the sensor can comprise a means of sensing the response of the smooth muscle to the electrical stimulation (the evoked response). The presence or absence of the evoked response can be used by the sphincter control system to evaluate if adequate stimulus intensity is being employed. These data can be logged to Memory so the clinician can evaluate any variations in stimulus threshold due to a changing medical condition, concomitant drug therapy or other change in the patient's condition. This automatically logged information can be combined with logged system events (for example, when the system is switched off then on when the patient wishes to urinate or defecate), to allow the clinician to review the use of the system by the patient.

The Measurements module may interface with the Sensor Processing module which as stated earlier, provides the specific circuitry for pre-conditioning or formatting of sensor data for processing by the Microprocessor. In addition, data used to assist in the control of the system may be collected and extracted and subsequently logged as additional information that can assist the clinician in managing the patient.

The stimulator also includes a Telemetry Interface 138 to transfer new values selected by the user to the Microprocessor or upload data from the implanted stimulator.

The microprocessor also interfaces with a Magnet Detection module 140, in the embodiment where a patient utilizes a magnet 142 to activate or de-activate the device. The magnet detection module provides control of the stimulator by the user or clinician. In the embodiment incorporating a battery, a magnet can be detected by the stimulator to provide a convenient means for the user to temporarily switch the system on or off or to code other functions that are required by the user.

In more detail, the placement of the magnet over the site of the implanted stimulator includes the following system functions:
Off—turn the system off while the magnet is continuously located over the implantable stimulator.
Toggle—the temporary presence of a magnet can toggle the system on to off, and the next presence from off to on.
Temporary Off—the temporary presence of a magnet can trigger the system to be programmed off for a programmable period of time.

For example, in the clinic environment a magnet can be utilized to turn the system off while the magnet is continuously located over the stimulator.

The stimulator also includes a circuit protector 108, which comprises electrical components which protect the implanted electronics from induced surge currents (e.g. during an external defibrillation pulse or diathermy).

Optionally, the circuit protector also includes filter circuitry to shield out electromagnetic interference. This is of particular use in embodiments where a sensing or sensor system is incorporated, as extraneous electromagnetic interference may confound signal processing.

As shown in FIGS. 1, 2, and 3, the sphincter control system is capable of communicating with an external controller 130A, 130B or 130C. The external controller, as previously described, can be used to provide a number of functions, including control of the sphincter, programming of the sphincter control system, or the downloading of diagnostic data.

In more detail, the external controller comprises a User Interface 144 as a means of providing input to the system to control the stimulation system. The User Interface 144 may include pushbuttons or a keypad to provide input and a visual display to allow confirmation of values or display of data or system status.

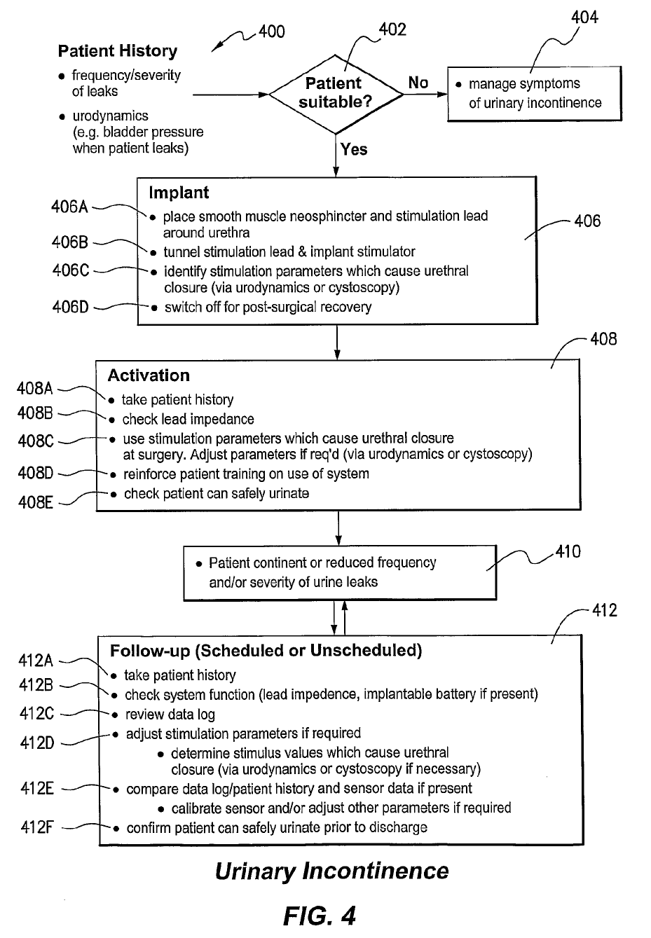

The external controller also includes a means 146 of converting the user instructions into the required data for transmission and/or control to the stimulator. The controller can provide pre-defined sequences that can simplify optimization of the system when operated by the clinician, or execute pre-defined sequences by the user. These sequences are often implemented in software. FIGS. 4, 5, 6 and 7 provide examples of control algorithms which can be used to optimize the function of an implantable system for stimulating a neosphincter.

Referring to FIG. 4, there is disclosed an example methodology for treating urinary incontinence-utilizing an embodiment of the present invention. Firstly, at 400, the patient history is taken, including data regarding to the frequency/severity of leakage of urine and also urodynamics (for example, the changes in the bladder pressure as the bladder is filled, the volume and pressure at time of leakage of urine from the bladder, and the changes in bladder pressure during urination). On the basis of this information, the suitability of the patient for an implant is determined. (402). If the patient is not suitable for an implant, conventional techniques (404) are utilized to manage symptoms of urinary incontinence.

If the patient is suitable for an implant, then the patient is scheduled for surgery for implant of a sphincter control system. The implant process (406) involves a number of sub steps. The first is to form a smooth muscle neosphincter around the urethra (406A) and attach a stimulation lead to the neosphincter. Next, the stimulation lead is “tunneled” to a stimulator, which is also implanted in the patient (406B). Thirdly, the surgeon verifies correct operation of the device by identifying the stimulation parameters which cause correct urethral closure (406C). This may be done via urodynamics (eg. filling the bladder and varying the stimulation of the smooth muscle neosphincter until closure is achieved) or by cystoscopy (that is, visually inspecting the area of the urethral meatus where the neosphincter is implanted, and looking to see if there is constriction and closure of the urethra on stimulation of the neosphincter).

The surgeon subsequently deactivates the implant to allow the patient to recover post-surgery (406D).

After the patient has been allowed to recover for a suitable amount of time (for example, two to four weeks), the patient undergoes an activation phase. Firstly, the medical professional takes a patient history (408A). If nothing untoward is discovered, the medical professional proceeds to check the lead impedance (408B).

After lead impedance has been checked, the neosphincter is stimulated to cause urethral closure, utilizing the parameters noted during surgery. The parameters are adjusted if required. (408C). The patient is then reminded how to use the system (408D). Lastly, the patient is checked to ensure that they can safely urinate (408E).

While the system is activated, the patient is preferably now continent, or at least, experiencing fewer and/or less severe leakages of urine (410).

The patient may also be asked to return for a follow up visit (or the patient may choose for one reason or another, to request a follow up visit) (412). During the follow up visit a patient history is taken (412A) secondly, a system function is checked, including a check of the lead impedance and the status of the implantable battery if present (412B). The data log of the device is also uploaded and reviewed (412C). After appropriate data has been collected, the stimulation parameters are adjusted if required (412D). Furthermore, the data log and patient history are compared and this information is used to calibrate the sensor(s) and/or adjust other parameters (412E).

Lastly, the patient is asked to perform the test to determine whether the patient can safely urinate prior to discharge (412F).

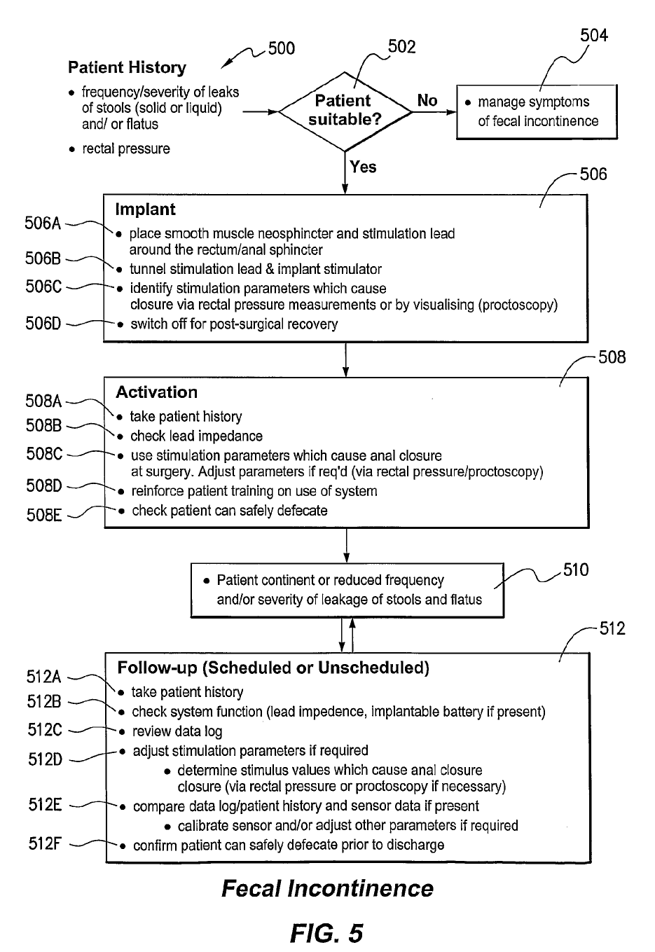

Referring to FIG. 5, there is disclosed an example methodology for treating fecal incontinence utilizing an embodiment of the present invention. Firstly, at 500, the patient history is taken, including data regarding to the frequency/severity of stools (solid or liquid) and flatus. In addition rectal pressure and anal closure pressure may be assessed to determine if the patient is suitable for implant. (502). If the patient is not suitable for an implant, conventional techniques (504) are utilized to manage symptoms of fecal incontinence.

If the patient is suitable for an implant, then the patient is scheduled for surgery to implant a sphincter control system for fecal incontinence. The implant process (506) involves a number of sub steps. The first is to form a smooth muscle neosphincter and attach a stimulation lead to it (506A). Next, the stimulation lead is “tunneled” to a stimulator, which is also implanted in the patient (506B). Thirdly, the surgeon verifies correct operation of the device by identifying the stimulation parameters which cause adequate closure (506C). This may be done by assessing rectal and or anal closure pressure whilst varying the stimulation of the smooth muscle neosphincter until closure is achieved or by proctoscopy (i.e. visually inspecting the anus and/or rectal passage for adequate closure).

The surgeon subsequently deactivates the implant to allow the patient to recover post-surgery (506D).

After the patient has been allowed to recover for a suitable amount of time (for example, two to four weeks), the patient undergoes an activation phase. Firstly, the medical professional takes a patient history (508A) if nothing untoward is discovered, the medical professional proceeds to check the lead impedance (508B).

After lead impedance has been checked, the neosphincter is stimulated to cause closure, utilizing the parameters noted during surgery. The parameters are adjusted if required. (508C). The patient is then reminded on how to use the system (508D). Lastly, the patient is checked to ensure that they can safely defecate (508E).

With the system on, the patient is preferably now continent, or at least experiences fewer and/or less severe leaks of stools (solid or liquid) and/or flatus (510).

The patient may also be asked to return for a follow up visit (or the patient may choose for one reason or another, to request a follow up visit) (512). During the follow up visit a patient history is taken (512A) secondly, a system function is checked, including a check of the lead impedance and the status of the implantable battery check if present (512B). The data log of the device is also revealed (512C). After appropriate data has been collected, the stimulation parameters are adjusted if required (512D). Furthermore, the data log and patient history are compared, and this information is used to calibrate the sensor(s) if present and/or adjust other parameters (512E).

Lastly, the patient is asked to perform the test to determine whether the patient can safely defecate prior to discharge (512F).

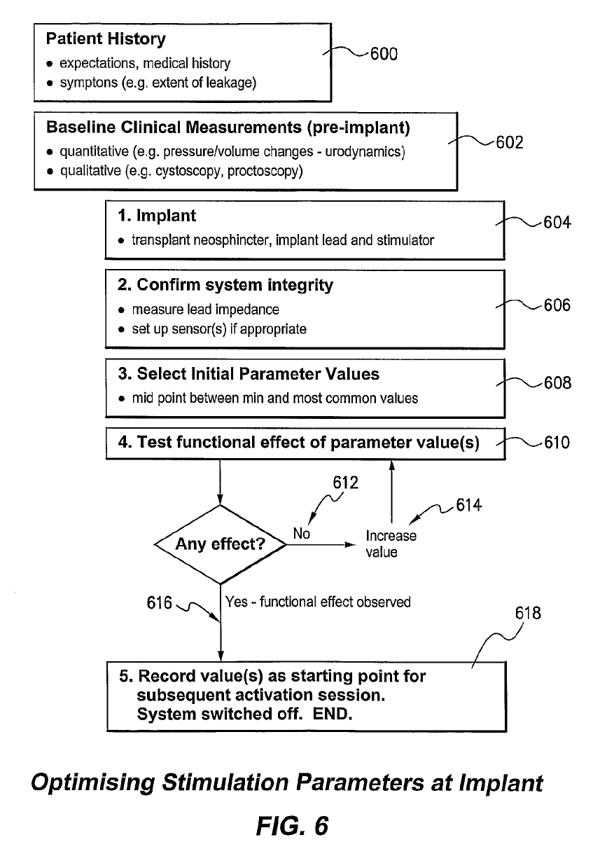

Referring to FIG. 6, there is provided a flowchart which outlines the methodology for optimizing the stimulation parameters of a device in accordance with an embodiment of the present invention. FIG. 6 is directed to optimising stimulation parameters at implant. At 600, a patient history is taken, to determine the presence of other medical conditions, to determine the expectations of the patient, and also to determine the extent of leakage or any other problems associated with bladder and/or bowel function.

Further, at 602, a series of baseline clinical measurements are taken, such as information regarding pressure/volume changes, and also tests which provide visual information on the closure of the patient's relevant sphincter (for example a cystoscopy or a proctoscopy) prior to implant of a sphincter control system.

Once this information is provided, a patient may then be implanted with a neosphincter, stimulation lead and implantable stimulator (604). Once the implant has been placed in the patient, before surgery is concluded, the surgeon confirms the system integrity by measuring the lead impedance and setting up any appropriate sensors (606). Initial values are generally taken as the midpoint between the minimum and the most common values (608) to commence investigation as to whether the electrical stimulation has the desired effect (610) If there is no affect (612) the value is increased (614) and a functional test is re-performed (610). This process is repeated until a functional effect is observed (616). Once a functional affect is observed the values are record as the starting point for subsequent activation (618) which typically occurs two to four weeks later to allow for healing and recovery of the patient. Should no functional effect be observed, the surgical team may consider further tests to evaluate the system, or alternatively record the maximum value for the parameter.

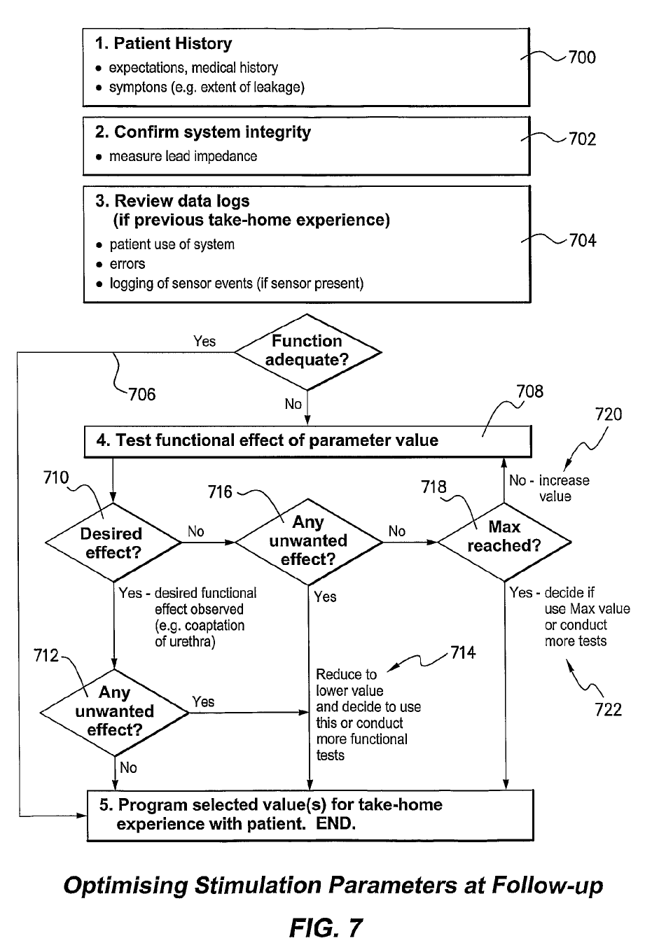

Referring to FIG. 7, there is provided a general description for optimising stimulation parameters at a follow up visit. Firstly, at 700 a patient history is taken. Secondly at 702, the lead impedance is measured to confirm the system integrity as well as other measurements such as the status of the implantable battery if present. At 704, data logs are reviewed to determine the patient use of the system, as well as whether any errors have been logged and the presence of any sensor events (if a sensor is present). If the patient is content and considers the function of the system is adequate, and the clinician considers there is no need to vary the programming of the system, no further action need be taken (706). If the function is not adequate, a functional test of the parameter values is performed (708). If the desired effect is present (710) then a test is performed to determine whether any unwanted side effects are also present (712). This testing cannot be readily conducted at implant as some unwanted effects may only be perceived by the patient when they are conscious (compared with under a general anaesthetic or perhaps heavily sedated during the surgery to implant the system). If no unwanted side effects are present, then no further action need be taken. If there is an unwanted effect, the parameter values are reduced to a lower value and a decision is made as to whether to conduct more functional tests (714). If no further functional tests are required then no further action need be taken. Returning to step 710, if no desired effect is found after performing a functional test, a test is performed to determine whether any unwanted effects are present (716). If unwanted effects are present, the parameter values are reduced (714). If no unwanted effects are present, and providing the parameter value maximum has not been reached (718), the value is increased (720) and a further functional effect test is performed (708).

If the maximum value has been reached, then a decision is made as to use the maximum value or whether to perform further functional tests (722).

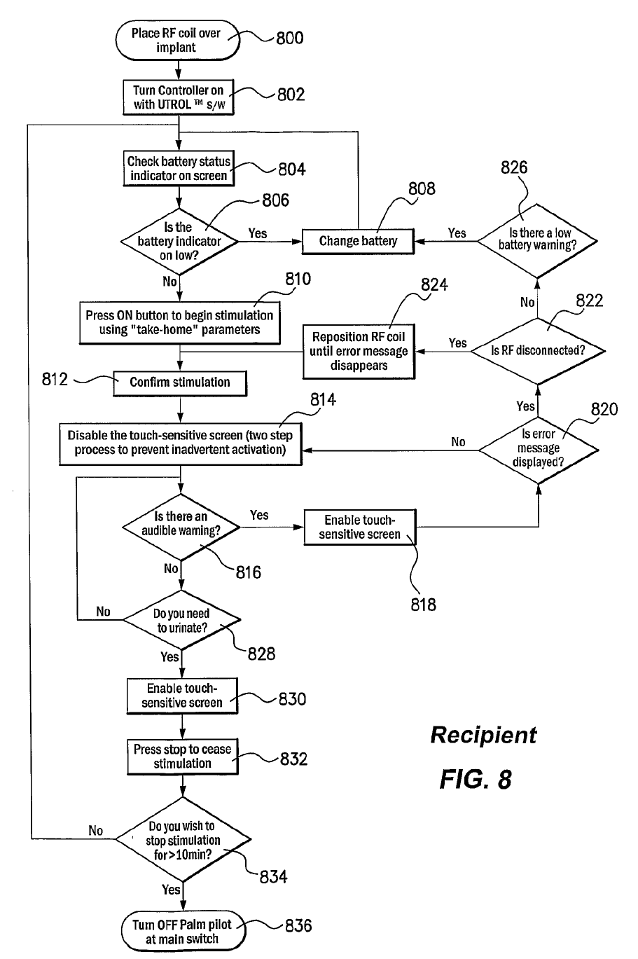

Referring to FIG. 8, there is disclosed an example control algorithm for the embodiment which includes an RF coil. FIG. 8 outlines a procedure which would be followed by a patient. As a first step 800, the patient would place the RF coil over the implantable stimulator system (implant). This would cause the controller to activate, as shown at step 802. In this embodiment, an external controller is used to set up the parameters necessary for a direct operation of the control system. Therefore, firstly, the battery status is checked at 804. If the battery indicator is on low (806) then the patient or the clinician would change the battery 808 and return to step 804. If the battery is not low, the test proceeds to step 810 where the “ON” button is pressed to begin stimulation using “take home parameters”. Once the “ON” button is pressed, stimulation is confirmed at step 812, and the touch sensitive screen on the external controller is subsequently disabled at step 814. This should precipitate an audible warning from the system. As shown at step 816 if there is an audible warning, the touch sensitive screen is enabled at step 818 and a check is made (step 820) to determine whether an error message is displayed. If no error message is displayed, the system returns to step 814, where the touch sensitive screen is disabled.

If an error message is displayed, a check is made (822) to determine whether the RF coil is disconnected. If the RF coil is disconnected, the algorithm proceeds to step 824 where the RF coil is repositioned until the error message disappears, at which the algorithm can return to step 812 where stimulation is confirmed.

If the RF coil is not disconnected, the algorithm proceeds to step 826 where a test is made to determine whether there is a low batter warning. If there is a low battery warning, the algorithm returns to step 808, where the clinician or the patient is required to change the battery, at which point the algorithm returns to the earlier step 804 of checking the battery status.

Returning to step 816, if there is no audible warning, then the patient must determine whether they need to urinate (step 828). If the patient does not need to urinate, the algorithm returns to step 816. If the patient needs to urinate, then the touch sensitive screen is enabled (step 830) and the “STOP” button is pressed to cease stimulation (step 832). Subsequently, a determination must be made as to whether the patient wishes to stop chronic stimulation and cease using the system for an extended period (for example, greater than 10 minutes) (834). If so, to conserve battery life, the algorithm proceeds to step 836 where the external controller is turned off. If not, the algorithm returns to step 804 and resumes the ordinary cycle of events, beginning with a check of the battery status indicator at step 804.

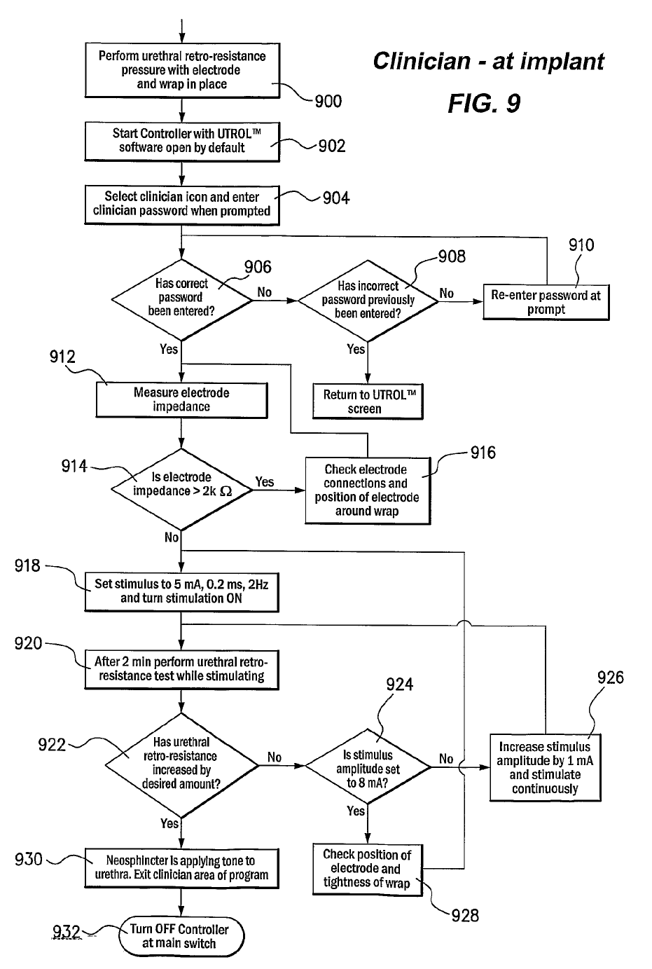

FIG. 9 describes the set-up procedure carried out by the clinician at the time the implant is placed in the patient. At step 900, with the stimulation lead and neosphincter in place, the clinician measures pressure changes along the urethra, including in the area of placement of the neosphincter, by drawing a catheter slowly along the urethra, (this test is known as a urethral pressure profile). The urethral pressure profile provides an indication of the tone generated by the neosphincter in response to a given stimulus level. Cystoscopy, in which the clinician uses a cystoscope to look at the inner surface of the urethra and/or sphincter, may be used as another diagnostic test. This can provide feedback on the stimulus level at which a functional change in diameter of the urethra is first observed in response to neosphincter stimulation, and provide confirmation that closure has been achieved with the selected stimulation parameters.

The external controller is then activated at step 902 and the appropriate software is loaded into memory. Subsequently, at step 904, the clinician enters a password to allow the clinician access to functionality which is generally only available to the clinician (and not to the patient). At step 906 a test is made to determine whether a password is correctly entered. If the password is not correctly entered, and an incorrect password has previously been entered, then the device returns to the set-up screen. Alternatively, no incorrect password has previously been entered, the clinician is given a further opportunity to re-enter the password at step 912, at which point the password is verified and the algorithm returns to step 906.

If the correct password has been entered, the first diagnostic test performed by the external controller is to measure the electrode impedance at step 914. If the electrode impedance is greater than 2 kilo-ohms (step 916), then the clinician must check the electrode connections and the position of the electrode around the neosphincter (step 918) at which time the electrode impedance must be re-measured (the algorithm returns to step 914).

If the stimulation lead impedance is not greater than 2 kilohms, the clinician may then set the stimulus to 5 mA 0.2 milliseconds, 2 Hz and subsequently turn the simulation on (step 920), as a convenient starting point to assess the effect of stimulation parameters on the bodily function (here, urethral closure by stimulation of a neosphincter). Subsequently at step 922 the urodynamics test is repeated while the neosphincter is stimulated. Subsequently, a test is performed to determine whether the urethral pressure profile has increased by an acceptable amount (step 924). If the urethral pressure profile has not increased by a suitable amount, a check is made to determine whether the stimulus amplitude is set to maximum (shown as 8 mA (step 926) in this example). If not, the stimulus amplitude is increased by 1 mA and the stimulation is continuously applied (step 928) at which time the algorithm returns to step 922 to re-assess function by an urodynamics test. If the stimulus amplitude is set to 8 mA, with no functional change, then a check is made to determine whether the position of the electrode is correct and the positioning of the neosphincter is correct (step 930). The algorithm then returns to step 920, where the test is begun again.

Returning to step 924, if the urethral pressure profile has increased by a correct amount, then the implant is working correctly. Therefore, the clinician can exit the clinician area of the program (step 932). As the procedure is then complete, the external controller may be turned “OFF” at the mains switch (step 934).

Other diagnostic tests are also feasible (for example, visual inspection of the meatus of the urethra via cystoscopy), to assess the system's function.

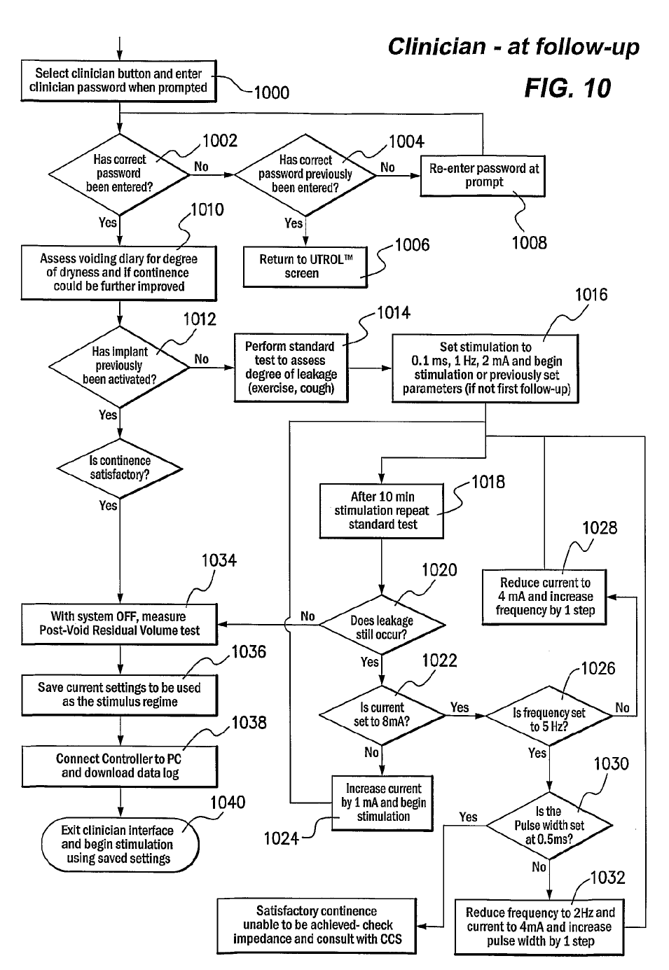

Referring now to FIG. 10, there is shown a flowchart which describes a typical procedure (algorithm) followed by a clinician at follow-up to implant of a device in accordance with any one of the embodiments described herein.

At step 1000, the clinician selects the clinician icon on the external controller and enters the clinician password when prompted (step 1000). A test is carried out to determine whether the correct password has been entered (step 1002). If an incorrect password has been entered, a check is made to determine whether an incorrect password has been previously entered (step 1004). If so, the clinician is returned to the main screen of the external controller (step 1006). If not, the clinician is prompted to re-enter the password (step 1008).

If the correct password has been entered, the clinician may then access and assess the voiding diary to make an assessment as to the degree of dryness and if continence could be further improved (step 1010).

This process is begun by determining whether the system has been previously activated (step 1012). If not, a standard test is performed to assess the degree of leakage (step 1014). Subsequently, stimulation is set to a convenient starting point to assess the effect of stimulation (for example, 1 millisecond, 1 Hz, 2 mA, or any other combination that a clinician may consider appropriate given the patient history) and the stimulation restarted (step 1016). Subsequently, at step 1018 after 10 minutes of simulation then standard test is repeated or valsalva leak point pressure test is initiated (a “worst case” test of the pressure at which urine leakage occurs when the patient voluntarily increases their intra-abdominal pressure by contracting the diaphragm with a closed glottis, thereby increasing the pressure on the bladder). A test is then carried out to determine whether leakage still occurs or whether the valsalva leak point pressure is low (step 1020). If the pressure is low, and the current is not set to 8 mA (step 1022), the maximum output in this example, the current is increased by 1 mA and the stimulation is restarted (step 1024). Subsequently, the algorithm returns to step 1018.

If the current is set to 8 mA and the frequency is set to less than 5 Hz (step 1026), the current is reduced to 4 mA and the frequency is increased by one step (step 1028), after which the algorithm returns to step 1018. If the frequency is set to 5 Hz and the pulse is set to less than 0.5 milliseconds (step 1030), the frequency is reduced to 2 Hz and the current is reduced to 4 mA and the pulse width is increased by one step (step 1032). Subsequently, the test returns to step 1018.

Returning to step 1020, if there is no leakage, the system proceeds to step 1034 where the post void residual volume is measured while the system is off. Subsequently, the current settings are saved and will be used as the “take home stimulus regime” (step 1036).

Subsequently, the controller is connected to the PC and the data log is downloaded (step 1038). The clinician can then exit the software and begin stimulation using the saved settings (step 1040).

The controller also includes a Telemetry Interface to code the data for transmission or decode data sent from the stimulator. There is also provided a power source, which may be a battery in the case where the controller is designed to be portable. Alternatively, the external controller may be arranged to be essentially a stationary device (e.g. where it is to be used mainly by the clinician for diagnostic and programming purposes). In this case, the power supply may be a mains power supply.

The external controller is optionally capable of interfacing with a computing system, to facilitate data management and analysis (for example, the external controller may have a communication means such as a USB or RS232 port, an infra-red or Bluetooth port, or any other suitable communication means). In one embodiment, the external controller is a PCI card arranged to be connected directly to the motherboard of a personal computer.

Where the stimulator is utilized to stimulate a neosphincter (for example, in a bodily function that is controlled by the Autonomic Nervous System in which typically there is no conscious sensation) the user has no conscious perception of the operation of the implanted system.

Therefore, the stimulator system includes additional cues to confirm operation of the system. The cues may take any one of a number of forms, including:
A means to provide a tactile sensation to the patient—a temporary variation in the stimulation mode (e.g. from bipolar to unipolar to the case of the stimulator) and/or intensity and/or frequency can be used to provide a perceived sensation to the patient when the system is initially switched on or a means of mechanically vibrating the implant itself
An audible cue—the stimulator system may include an audible sound to alert the patient that the magnet has changed state of operation of the system; and
A visual cue—the stimulator system may include a visual indicator such as a small Light Emitting Diode that triggers when a pulse is delivered by the stimulator.

Any or all of these means may be utilized to indicate to the patient a change in the function of the stimulator.

The addition of a sensor to the sphincter control system allows for the automation of various functions associated with incontinence. For example, a sensor may be utilised to detect excessive bladder volume and trigger an alarm to warn the patient that they should urinate at a socially convenient time.

Another sensor can be utilized to detect the commencement of urination or defecation and automatically switch off stimulation to the neosphincter (“void initiate”). The same sensor, or a different sensor, can be utilized to determine when voiding has finished, to automatically restart the stimulation after the user completes the voluntary act (“void complete”). The “void initiate” and “void complete” functions, when driven by a sensor, reduce the need for an external controller. However, there may be situations where the patient prefers to also have an external controller.

The stimulator previously described may also be arranged to train muscles which have inadequate function, for example the pelvic floor muscles can also be important in the maintenance of continence during a sudden pressure change, such as a cough or a sneeze. The presence of electrical stimulation can influence the re-innervation of denervated skeletal muscle. Electrical stimulation has the potential to modify the density or orientation of innervation to provide superior function and may assist in the return of autonomous function of smooth muscle.

The stimulator may be utilized to restore control of incontinence through the temporary application of electrical stimulation of a free graft smooth muscle using an implanted sphincter stimulator. The embodiments for a stimulator described in the present application may be appropriate for such an application and may necessitate the further step of stimulating the transplanted of the smooth muscle tissue immediately following surgery to encourage specific density or orientation of innervation. In this instance, it may be that continence is achieved without the need for stimulation of the transplanted smooth muscle.

In an alternate embodiment, there may also be provided a system which utilizes stimulation leads that pass from a neosphincter percutaneously to an external stimulator that enables the delivery of temporary application of electrical stimulation of a free graft smooth muscle.

In either of the embodiments utilized for controlling incontinence, there is optionally provided a sensor to provide a female patient with feedback on the effectiveness of training of pelvic floor muscles. The sensor can provide such feedback through the measurement of generated changes in bladder pressure by contraction of the pelvic floor.

Advantages of the embodiments described herein include the activation of a discrete piece of smooth muscle tissue that is transplanted or placed to overcome a deficiency in a bodily function and which can then can be activated by the stimulator, algorithms to optimize the selection of parameters to efficiently stimulate the smooth muscle tissue and means of conveying to the user or supervisory clinician the effect of this stimulation—either by direct measurement of clinical parameters (for example, the volume of urine leaked or the user's perception of fullness of the bladder) and the use of data logging and feedback to optimise the values of operating parameters for the stimulation of smooth muscle tissue.

It will be understood that whilst the embodiments described herein refer to a controller for a single sphincter, each of the described embodiments may be arranged to operate multiple sphincters. The controller may be arranged to contain multiple outputs and to control multiple sphincters in response to commands from a central control unit.

The claims defining the invention are as follows:

1. A device for the stimulation of smooth muscle tissue, comprising a stimulator arranged to provide a signal to the smooth muscle tissue to control response of the smooth muscle tissue, and an interface arranged to allow programming of a controller of the stimulator.

2. A device in accordance with claim 1, wherein the signal is passed from the stimulator to the smooth muscle tissue via a stimulation lead.

3. A device in accordance with claim 2, wherein the stimulation lead has at least one electrode to deliver electrical stimulation to the smooth muscle tissue.

4. A device in accordance with any one of the preceding claims, wherein the smooth muscle tissue is one of a sphincter and a neosphincter.

5. A device in accordance with any one of the preceding claims wherein the signal delivered to the smooth muscle tissue is a symmetric or an asymmetric wave form.

6. A device in accordance with claim 5, wherein the wave form is a biphasic wave form.

7. A device in accordance with claim 6, wherein the stimulator is arranged to apply a delay between two phases of the biphasic wave form.

8. A device in accordance with claim 7, wherein the delay between the two phases is in the range from approximately 0 to 100 milliseconds.

9. A device in accordance with any one of the preceding claims, wherein the signal is an electric signal with a current amperage in the range of zero to twenty-five milliamps in each phase.

10. A device in accordance with claim 1, claim 2 or claim 3 wherein the impedance of the stimulation lead is in the range of approximately 300 ohms to 2000 ohms.

11. A device in accordance with any one of the preceding claims, wherein the device is implantable within a patient.

12. A device in accordance with any one of the preceding claims, further comprising at least one sensor.

13. A device in accordance with claim 12, wherein the sensor is arranged to monitor the response of the smooth muscle tissue.

14. A device in accordance with claim 12, wherein the at least one sensor is arranged to monitor the response of a bodily function of the patient.

15. A device in accordance with claim 14, wherein the bodily function is the fullness of the bladder of the patient.

16. A device in accordance with claim 14, wherein the bodily function is the fullness of the rectum of the patient.

17. A device in accordance with any one of claims 12 to 16, further comprising storage means arranged to store data collected from the at least one sensor.

18. A device in accordance with any one of the preceding claims, further including a power source in the form of an in-built battery.

19. A device in accordance with claim 17, wherein the battery is rechargeable.

20. A device in accordance with any one of claims 1 to 12, further including a power source in the form of a charge storing device.

21. A device in accordance with claim 20, wherein the charge storing device is a capacitor.

22. A device in accordance with any one of the preceding claims, further including a switch arranged to control stimulation of the device.

23. A device in accordance with claim 22, wherein the switch is a magnetic switch.

24. A device in accordance with claim 22, wherein the switch is a wireless communication device capable of sending an electromagnetic signal to the device.

25. A device in accordance with any one of the preceding claims, further comprising means to provide feedback on the status of the device.

26. A device in accordance with claim 25, wherein the feedback is provided by a tactile signal, such as a vibration.

27. A device in accordance with claim 25, wherein the feedback is provided by an audible signal.

28. A device in accordance with claim 25, wherein the feedback is provided by a visual signal.

29. A device in accordance with any one of claims 25 to 28, wherein the feedback includes information on the power status of the device.

30. A device in accordance with any one of claims 25 to 29, wherein the feedback includes information on the power level of the device.

31. A device for the stimulation of smooth muscle tissue, comprising a stimulator arranged to provide a signal to the smooth muscle tissue to control response of the smooth muscle tissue, and means to provide feedback to a patient on the status of a controller for the stimulator.

32. A device in accordance with claim 31, wherein the feedback is provided via a tactile signal, such as a vibration.

33. A device in accordance with claim 31 or 32, wherein the feedback is provided by an audible signal.

34. A device in accordance with any one of claims 31 to 33, wherein the feedback is provided by a visual signal.

35. A device in accordance with any one of claims 31 to 34, wherein the feedback includes information on the power status of the device.

36. A device in accordance with any one of claims 31 to 35, wherein the feedback includes information on the power level of the device.

37. A device in accordance with any one of claims 31 to 36, wherein the feedback includes information on the fullness of the bladder or rectum.

38. A device for use with a stimulator arranged to provide a signal to smooth muscle tissue to control response of the smooth muscle tissue, the device comprising at least one sensor arranged to monitor a bodily function of a patient.

39. A device in accordance with claim 38, wherein the bodily function is the fullness of the bladder of the patient.

40. A device in accordance with claim 38, wherein the bodily function is the fullness of the rectum of the patient.

41. A device in accordance with claim 38, wherein the bodily function is the commencement and/or cessation of urination by the patient.

42. A device in accordance with claim 38, wherein the bodily function is the commencement and/or cessation of defecation by the patient.

43. A sensor arranged to provide feedback of a bodily function associated with one of a sphincter and a neosphincter.

44. A system for the stimulation of smooth muscle tissue, comprising a device in accordance with any one of claims 1 to 43 and an external controller arranged to communicate with the device.

45. A system in accordance with claim 44, wherein the external controller is arranged to program the stimulator controller.

46. A system in accordance with claim 44 or claim 45, wherein the external controller is arranged to upload data to a storage means.

47. A system in accordance with claim 44, claim 45 or claim 46, wherein the external controller is arranged to download data from a storage means.

48. A device for the stimulation of smooth muscle tissue, comprising a stimulator arranged to provide a signal arranged to stimulate the smooth muscle tissue, and a controller arranged to control the signal provided by the stimulator in a manner which influences the innervation of the smooth muscle tissue.

49. A method for calibrating a device for the stimulation of smooth muscle tissue, comprising the steps of measuring the impedance of a stimulation lead arranged to provide a stimulus signal to the smooth muscle tissue, measuring the response of the smooth muscle tissue, and, if necessary, adjusting the signal.

50. A method of stimulating a smooth muscle tissue, comprising the steps of utilising a device in accordance with claim 1 to control the smooth muscle tissue, by applying a signal to the smooth muscle tissue to cause the smooth muscle tissue to contract.

51. A method for stimulating the pelvic floor, comprising the steps of utilising a device in accordance with claim 1 to stimulate the pelvic floor to cause the pelvic floor muscle to contract, thereby strengthening the pelvic floor.

52. A device in accordance with any one of claims 1 to 43, comprising a plurality of stimulators to stimulate a plurality of discrete smooth muscle neosphincters.

53. A device in accordance with claim 51, wherein the plurality of bodily functions includes at least one of the control of urine flow from the bladder and the control of stools and/or flatus from the rectum.
